(19) United States
(12) Patent Application Publication
(10) Pub. No.: US 2010/0240116 A1
(43) Pub. Date: Sep. 23, 2010

(54) LOVASTATIN ESTERASE ENZYME IMMOBILIZED ON SOLID SUPPORT, PROCESS FOR ENZYME IMMOBILIZATION, USE OF IMMOBILIZED ENZYME, BIOCATALYTIC FLOW REACTOR AND PROCESS FOR PREPARATION AND/OR PURIFICATION OF SIMVASTATIN

(75) Inventors: Ryszard Ostaszewski, Pecice Male (PL); Dominik Koszelewski, Warszawa (PL); Waldemar Kurek, Warszawa (PL); Dorota Patralska, Siedlce (PL)

Correspondence Address: MARSH, FISCHMANN & BREYFOGLE LLP, 8055 East Tufts Avenue, Suite 450, Denver, CO, 80237, US

(73) Assignee: INSTYTUT CHEMII ORGANICZNEJ, POLSKA AKADEMIA NAUK, WARSZAWA (PL)

(21) Appl. No.: 12/743,040

(22) PCT Filed: Nov. 18, 2008
(86) PCT No.: PCT/PL08/00086

§ 371 Date: May 14, 2010

(30) Foreign Application Priority Data
Nov. 19, 2007 (PL) P 383819

(51) Int. Cl. C12P 17/06; C12N 9/96; C12M 1/00

(52) U.S. Cl. 435/280; 435/188; 435/289.1

(57) ABSTRACT

The invention relates to the lovastatin esterase enzyme immobilized on a solid support insoluble in water, the enzyme being covalently bound to a solid support activated with an at least difunctional coupling reagent, the immobilized lovastatin esterase exhibiting at least 5 times higher the hydrolytic activity towards lovastatin and salts thereof, in the presence of simvastatin and/or salts thereof, than towards simvastatin and salts thereof. The invention also relates to a process for immobilization of the lovastatin esterase enzyme on a solid support insoluble in water, and use of the enzyme immobilized on a solid support for preparation and/or isolation and/or purification of simvastatin, and also to a process for preparation and/or purification of simvastatin comprising treating the solution of the simvastatin salt containing residual content of the lovastatin salt with the lovastatin esterase enzyme until hydrolysing lovastatin to form the triol; separating the triol; and isolating simvastatin substantially free from lovastatin, wherein the solution of the simvastatin salt containing residual content of the lovastatin salt, is brought into a contact with the lovastatin esterase enzyme immobilized on a solid support insoluble in water. Also, the invention relates to a biocatalytic flow reactor with a bed, comprising a body (1) of the reactor with an inner space (2) connected to the fluid inlet (3) and connected to the fluid outlet (4), in which inner space (2) there is a bed (5) containing the lovastatin esterase enzyme immobilized on a solid support insoluble in water.

This invention relates to the lovastatin esterase enzyme immobilized on a solid support insoluble in water, a process for an enzyme immobilization, use of an immobilized enzyme, a biocatalytic flow reactor and a process for preparation and/or purification of simvastatin.

Simvastatin and lovastatin belong to the class of compounds being the hydroxy-3-methyl-glutaryl-coenzyme A (HMG-CoA) reductase inhibitors, named statins. Statins significantly reduce risk of coronary arterial disease, cerebral stroke and peripheral atherosclerosis. By reduction of cholesterol deposits, they stabilise the atheromatous plaque, improve the function of vascular endothelium, inhibit growth and migration of smooth muscle cells, and also have a beneficial effect on blood clotting, fibrinolysis, and the activity of platelets, and also exhibit an anti-inflammatory effect [Tobert J. A. et al., Journal of Clinical Investigations, 1982, April, 69 (4), 913-919; Pedersen T. et al., Lancet (1994) 344, 1383-89; Czynniki Ryzyka. Kwartalnik Polskiego Towarzystwa Badań nad Miażdżyc [symbol] (Risk Factors. Polish Society of Atherosclerosis Research Quarterly) 2003, 40, 5-13].

Lovastatin, the compound of the formula I, is prepared via fermentation using the Aspergillus terreus strain. Simvastatin, the compound of the formula II, having a higher pharmacological activity and a lower toxicity than lovastatin, is prepared semi-synthetically from lovastatin.

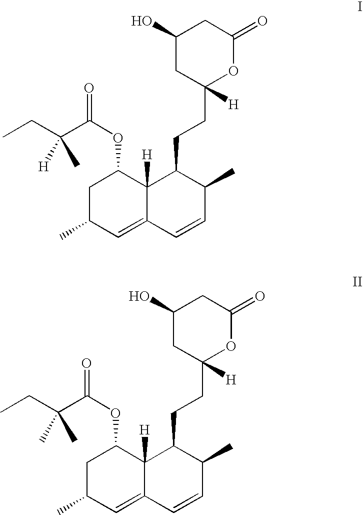

U.S. Pat. No. 4,582,915 discloses a process for preparing simvastatin that consists in modifying the side chain of lovastatin, according to the following scheme.

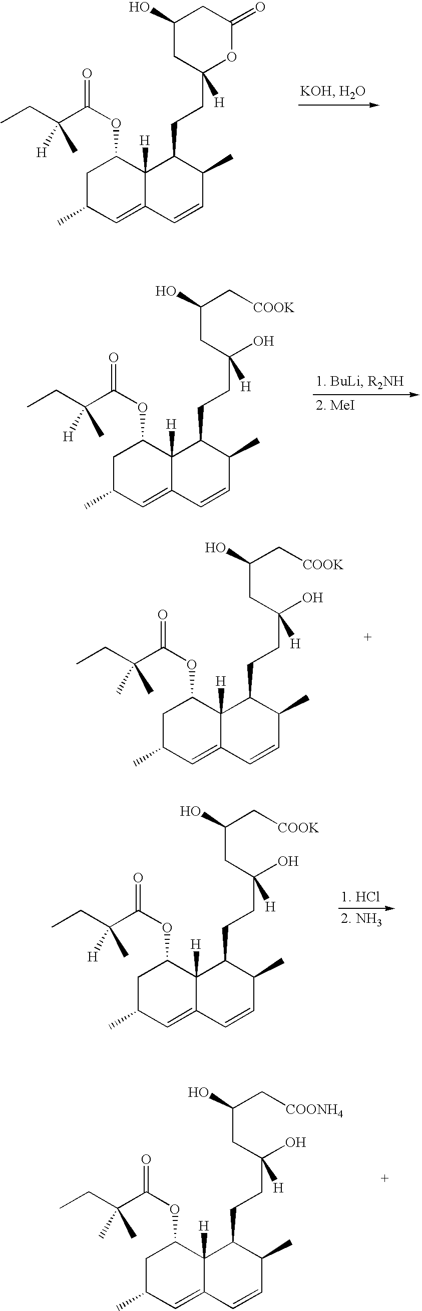

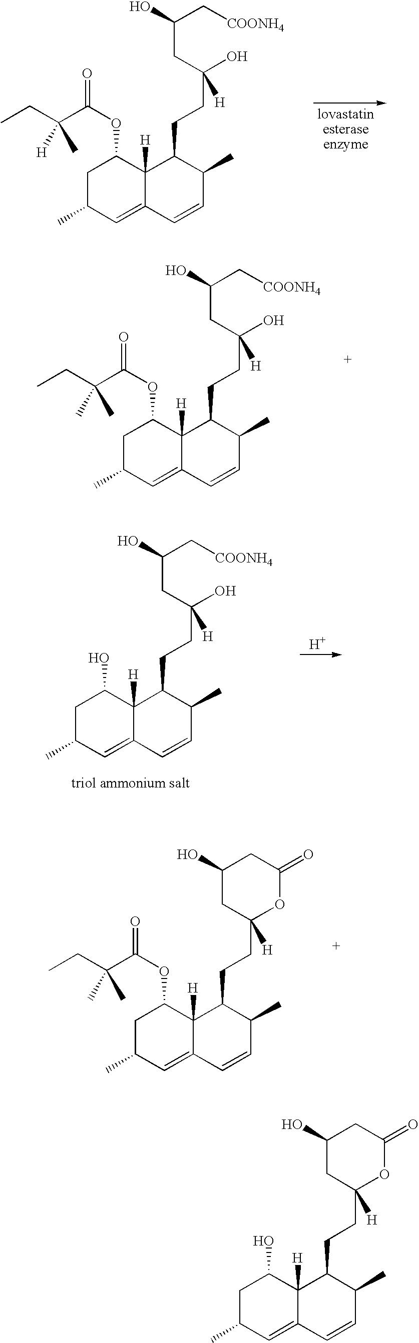

Lovastatin is subjected to the alkaline hydrolysis, and the formed potassium salt is treated with a strong base such as n-butyllithium in the presence of a secondary amine, followed by a methylating agent (e.g., methyl iodide). The resulting conversion does not exceed 95%. Isolation of simvastatin in its pure form is difficult because the contamination with residual lovastatin can be separated, in principle, only with the use of HPLC.

The lovastatin esterase enzyme makes it possible to selectively hydrolyse lovastatin salts in the presence of simvastatin salts. This enzyme is produced by the hyphae-forming fungus Clonostachys compactiuscula (ATCC 38009, ATCC 74178). Following the conversion into ammonium salts, the mixture of simvastatin and lovastatin is subjected to enzymatic hydrolysis reaction catalyzed with lovastatin esterase, that results in selectively hydrolysing the lovastatin salt into the “triol”, leaving the simvastatin salt untouched. The lactonisation of the mixture of acids in acidic conditions leads to the formation of a mixture of lactones that may be separated by crystallization.

Performing the enzymatic hydrolysis reaction using the native lovastatin esterase enzyme results in a loss of the enzyme, what increases the costs of transforming lovastatin into simvastatin. The use of a whole-cell material from the culture of Clonostachys compactiuscula is possible but this complicates the isolation of the product.

The immobilization of the enzyme on a solid support makes it possible to avoid these inconveniences. Attempts to immobilize the lovastatin esterase on a solid support insoluble in water were disclosed by Ostaszewski R. et al., Biotechnology (2006, 888-892). Nevertheless, the obtained catalysts presented low activity, and the immobilized enzyme was characterized by an absence or a significant reduction of the selectivity of hydrolysing lovastatin versus simvastatin, i.e., the enzymatic process resulted in hydrolysis of both lovastatin and simvastatin.

The object of this invention is to provide the lovastatin esterase enzyme immobilized on a solid support insoluble in water, a process for an enzyme immobilization, use of an immobilized enzyme, a biocatalytic flow reactor as well a process for preparation and/or purification of simvastatin.

The lovastatin esterase enzyme immobilized on a solid support insoluble in water, according to the invention, is characterized in that the enzyme is covalently bound to a solid support activated with an at least difunctional coupling reagent, the immobilized lovastatin esterase exhibiting at least 5 times higher the hydrolytic activity towards lovastatin and salts thereof, in the presence of simvastatin and/or salts thereof, than towards simvastatin and salts thereof.

Advantageously, the solid support is a polysaccharide or a modified polysaccharide. The polysaccharide is especially a polygalactoside. In particular, the modified polysaccharide is a di-(C₁₋₆alkyl)amino-C₁₋₆alkylcellulose, especially diethylaminoethylcellulose.

Advantageously, the solid support is a silica gel or a modified silica gel. In particular, the modified silica gel is a silica gel modified with amino-C₁₋₆ alkyl-tri(C₁₋₁₋₆ alkoxy)silane, especially an aminopropylsilanized silica gel.

Advantageously, the at least difunctional reagent activating the solid support is a compound of the formula

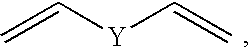

wherein Y represents —SO₂— or —SO₂—(CHR)ₙ—SO₂—, where n represents an integer of from 1 to 18, and R represents a hydrogen atom or C₁₋₆ alkyl, or Y represents —SO₂—Ar—SO₂—, where Ar represents a divalent aryl radical formed by displacing two hydrogen atoms directly bound to the aromatic ring carbon atoms, the divalent aryl radical optionally bearing C₁₋₆ alkyl substituents. In particular, the at least difunctional reagent activating the solid support is a compound of the formula

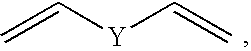

wherein Y represents —SO₂—.

Advantageously, the at least difunctional reagent activating the solid support is a cyanuric halide and/or cyanuric acid O-sulphonate. In particular, the at least difunctional reagent activating the solid support is a cyanuric halide, especially cyanuric chloride.

Advantageously, the solid support is a polygalactoside, and the at least difunctional reagent activating the solid support is the compound of the formula

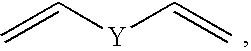

wherein Y represents —SO₂—.

Advantageously, the solid support is diethylaminoethylcellulose, and the at least difunctional reagent activating the solid support is cyanuric chloride.

Advantageously, the solid support is an aminopropylsilanized silica gel, and the at least difunctional reagent activating the solid support is cyanuric chloride.

In particular, the enzyme is an enzyme produced by Clonostachys compactiuscula ATTC 38009, ATCC 74178.

A process for immobilization of the lovastatin esterase enzyme on a solid support insoluble in water, according to the invention, is characterized in that using mechanical agitation, a cyanuric halide is contacted with a solid support in a solvent, the activated solid support is separated by filtration, the activated solid support is dried and suspended in an aqueous mixture containing the lovastatin esterase enzyme, until immobilization of the enzyme, the suspended material is separated by filtration, washed with a buffer and dried.

Advantageously, a polysaccharide or a modified polysaccharide is used as a solid support. In particular, the modified polysaccharide is a di-(C₁₋₆alkyl)amino-C₁₋₆alkyl-cellulose, especially diethylaminoethylcellulose.

Advantageously, a silica gel or a modified silica gel is used as the solid support. In particular, a silica gel modified with amino-C₁₋₆alkyl-tri(C₁₋₆alkoxy)silane, especially an aminopropylsilanized silica gel is used as the modified silica gel.

Advantageously, the cyanuric halide used is cyanuric chloride.

In particular, an autoclaved solid support is used.

Optionally, the mechanical agitation used is shaking.

Advantageously, the filtered material is washed with water prior to washing with a buffer.

In particular, the cyanuric halide is contacted with the solid support in the presence of a base and/or a buffer.

The enzyme-containing aqueous solution used is, especially, a protein fraction of the material extracted from Clonostachys compactiuscula ATTC 38009, ATCC 74178.

A process for immobilization of the lovastatin esterase enzyme on a solid support insoluble in water, according to the invention, is characterized in that the compound of the formula

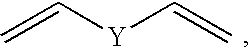

wherein Y represents —SO₂— or —SO₂—(CHR)ₙ—SO₂—, where n represents an integer of from 1 to 18, and R represents a hydrogen atom or C₁₋₆ alkyl, or Y represents —SO₂—Ar—SO₂—, where Ar represents a divalent aryl radical formed by displacing two hydrogen atoms directly bound to the aromatic ring carbon atoms, the divalent aryl radical optionally bearing C₁₋₆ alkyl substituents, is contacted with the solid polygalactose support in a solvent using mechanical agitation, the activated solid support is separated by filtration, the activated solid support is dried and suspended in an aqueous mixture containing the lovastatin esterase enzyme, the suspended material is separated by filtration, washed with a buffer and dried.

Advantageously, the compound of the formula

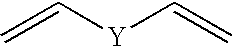

is a compound wherein Y represents —SO₂—.

In particular, shaking is used as a mechanical agitation.

Prior to washing with a buffer, the filtered material is washed optionally with water.

The enzyme-containing aqueous solution used is, especially, a protein fraction of the material extracted from Clonostachys compactiuscula ATTC 38009, ATCC 74178.

According to the invention, the lovastatin esterase enzyme immobilized on a solid support insoluble in water is used for preparation and/or isolation and/or purification of simvastatin.

Advantageously, the enzyme is used for purification and/or isolation of simvastatin from the mixture with lovastatin.

In particular, the enzyme is used for selectively hydrolysing the lovastatin ammonium salt into the triol salt, in the presence of the simvastatin ammonium salt. The enzyme is used, optionally, for selectively hydrolysing the lovastatin ammonium salt into the triol salt, in the presence of the simvastatin ammonium salt, in a batch process. The enzyme is used, especially, for selectively hydrolysing the lovastatin ammonium salt into the triol salt, in the presence of the simvastatin ammonium salt, in a continuous process.

A biocatalytic flow reactor with a bed according to the invention is characterized in that it comprises a body of the reactor with an inner space connected to the fluid inlet and connected to the fluid outlet, in which inner space there is a bed containing the immobilized lovastatin esterase enzyme.

Advantageously; the bed contains the enzyme immobilized on a solid support insoluble in water, the enzyme being covalently bound to the solid support activated with an at least difunctional coupling reagent.

In particular, the solid support is a polysaccharide or a modified polysaccharide. The polysaccharide is, especially, a polygalactoside. In particular, the modified polysaccharide is a di-(C₁₋₆alkyl)amino-C₁₋₆alkylcellulose, especially diethylaminoethylcellulose.

Advantageously, the at least difunctional reagent activating the solid support is a compound of the formula

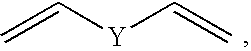

wherein Y represents —SO₂— or —SO₂—(CHR)ₙ—SO₂—, where n represents an integer of from 1 to 18, and R represents a hydrogen atom or C₁₋₆ alkyl, or Y represents —SO₂—Ar—SO₂—, where Ar represents a divalent aryl radical formed by displacing two hydrogen atoms directly bound to the aromatic ring carbon atoms, the divalent aryl radical optionally bearing C₁₋₆ alkyl substituents. In particular, the at least difunctional reagent activating the solid support is the compound of the formula

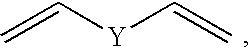

wherein Y represents —SO₂—.

Advantageously, the at least difunctional reagent activating the solid support is a cyanuric halide and/or cyanuric acid O-sulphonate. In particular, the at least difunctional reagent activating the solid support is a cyanuric halide, especially cyanuric chloride.

Advantageously, the solid support is a polygalactoside, and the at least difunctional reagent activating the solid support is a compound of the formula

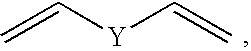

wherein Y represents —SO₂—.

Advantageously, the solid support is diethylaminoethylcellulose, and the at least difunctional reagent activating the solid support is cyanuric chloride.

The enzyme is an enzyme produced especially by Clonostachys compactiuscula ATTC 38009, ATCC 74178.

A process for preparation and/or purification of simvastatin comprising treating the solution of the simvastatin salt containing residual content of the lovastatin salt with the lovastatin esterase enzyme until hydrolysing lovastatin to form the triol, separating the triol, and isolating simvastatin substantially free from lovastatin, according to the invention, is characterized in that the solution of the simvastatin salt containing residual content of the lovastatin salt, is brought into a contact with the lovastatin esterase enzyme immobilized on a solid support insoluble in water.

Advantageously, the enzyme is covalently bound to the solid support activated with an at least difunctional coupling reagent.

Advantageously, the solid support is a polysaccharide or a modified polysaccharide. The polysaccharide is, especially, a polygalactoside. In particular, the modified polysaccharide is a di-(C₁₋₆alkyl)amino-C₁₋₆alkylcellulose, especially diethylaminoethylcellulose.

Advantageously, the solid support is a silica gel or a modified silica gel. In particular, the modified silica gel is a silica gel modified with amino-C₁₋₆alkyl-tri(C₁₋₆alkoxy)silane, especially an aminopropylsilanized silica gel.

Advantageously, the at least difunctional reagent activating the solid support is the compound of the formula

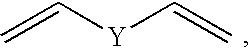

wherein Y represents —SO₂— or —SO₂—(CHR)ₙ—SO₂—, where n represents an integer of from 1 to 18, and R represents a hydrogen atom or C₁₋₆ alkyl, or Y represents SO₂—Ar—SO₂—, where Ar represents a divalent aryl radical formed by displacing two hydrogen atoms directly bound to the aromatic ring carbon atoms, the divalent aryl radical optionally bearing C₁₋₆ alkyl substituents. In particular, the at least difunctional reagent activating the solid support is the compound of the formula

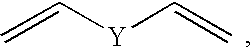

wherein Y represents —SO₂—.

Advantageously, the at least difunctional reagent activating the solid support is a cyanuric halide and/or cyanuric acid O-sulphonate. In particular, the at least difunctional reagent activating the solid support is a cyanuric halide, especially cyanuric chloride. Advantageously, the solid support is a polygalactoside, and the at least difunctional reagent activating the solid support is the compound of the formula

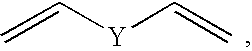

wherein Y represents —SO₂—.

Advantageously, the solid support is diethylaminoethylcellulose, and the at least difunctional reagent activating the solid support is cyanuric chloride.

Advantageously, the solid support is an aminopropylsilanized silica gel, and the at least difunctional reagent activating the solid support is cyanuric chloride.

In particular, contacting the solution of the simvastatin salt containing residual content of the lovastatin salt with the enzyme immobilized on a solid support insoluble in water is carried out at the temperature from 26° C. to 50° C.

Contacting the solution of the simvastatin salt containing residual content of the lovastatin salt with the enzyme immobilized on a solid support insoluble in water is carried out, especially, continuously in a flow reactor.

In particular, the enzyme is an enzyme produced by Clonostachys compactiuscula ATTC 38009, ATCC 74178.

Immobilization of the enzyme makes it possible to repeatedly reuse of the same enzyme without loss of its activity at the appropriately adjusted parameters of the process. What is extremely important, a resistance to the proteolytic degradation is enhanced, allowing to perform the process without using antiseptic conditions, thus lowering the costs significantly.

According to one embodiment of the invention, the immobilized enzyme is contained in a flow reactor. The reactor is supplied with a mixture of the statins comprising 15% lovastatin and 85% simvastatin. This is a typical mixture obtained in the chemical synthesis process. The concentration of the simvastatin salt does not change by more than 1%, whereas the lovastatin ammonium salt is hydrolysed in about 87%. The conversion level does not alter during the prolonged hydrolysis, and the technological stability is maintained for at least 6 months. Thus, the immobilization of the lovastatin esterase enzyme provides a stable biocatalyst exhibiting a stability sufficient for the technological application in the synthesis of simvastatin.

The technical solution according to the invention is illustrated in drawings, in which

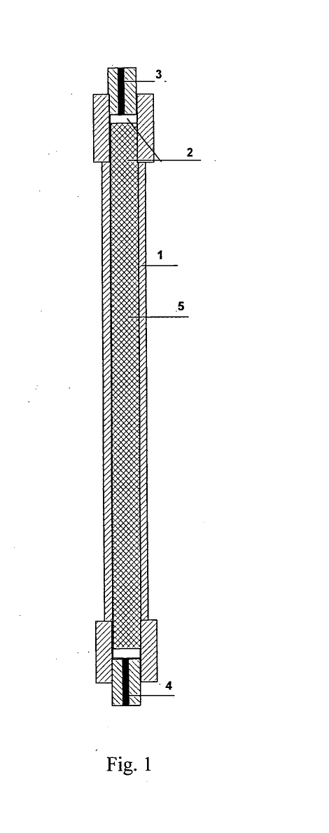

FIG. 1 presents a cross-sectional view of the biocatalytic flow reactor according to the invention, and

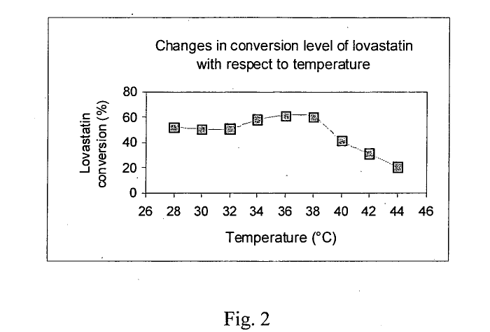

FIG. 2 presents changes in the conversion level of lovastatin with respect to temperature.

Throughout the description of this invention and the patent claims, the term “triol” denotes the compound of the formula III,

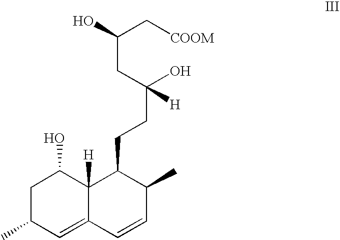

wherein M represents a hydrogen atom, and also wherein M represents a metal cation or ammonium cation, i.e., the compound in the free-acid form or in the salt form, respectively, if not specified otherwise. Since the compound III in the free-acid form (M=H) is easily lactonised, the term “triol” may also comprise the lactone-diol form of the formula IV, if not specified otherwise.

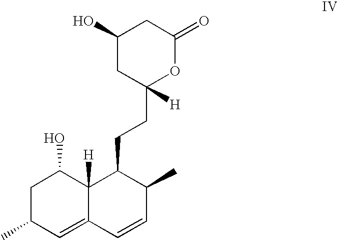

The names “lovastatin” and “simvastatin” refer to the compounds of the formulae I and II, respectively.

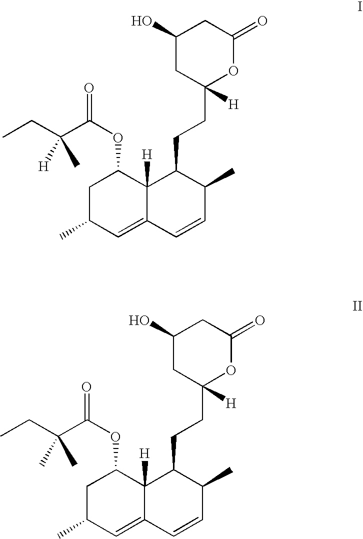

Throughout the description of this invention and the patent claims, the names “lovastatin” and “simvastatin” comprise also the carboxylic acid forms of these compounds, having the formulae IV and V (M=H), respectively,

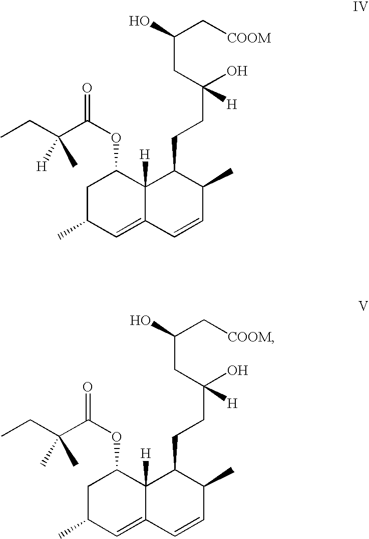

and also salts of the compounds of the formulae IV and V. if not specified otherwise. The “lovastatin salt” represents the compound of the formula IV, wherein M represents a metal cation or an ammonium cation, and the “simvastatin salt” represents the compound of the formula V, wherein M represents a metal cation or an ammonium cation.

Throughout the description of this invention and the patent claims, the “lovastatin esterase” denotes a cellular or non-cellular material of the natural or recombinant origin having an enzymatic activity that consists in catalysing the hydrolysis of lovastatin and salts thereof, to form the above-defined triol or salts thereof, where, under analogous conditions, the simvastatin salts do not undergo enzymatic hydrolysis or undergo enzymatic hydrolysis at a rate lower of at least one order of magnitude.

Throughout the description of this invention and the patent claims, the solid support insoluble in water denotes a granular or fibrous solid support, that principally does not undergo solubilisation in water, i.e., it does not form a liquid solution or pseudo-solution in water containing more than 0.1 g of the support per 100 g of water.

Throughout the description of this invention and the patent claims, the enzyme activity represents a micromolar amount of the substrate (lovastatin or simvastatin) that is hydrolysed within one minute with one milligram of the enzyme.

Throughout the description of this invention and the patent claims, the protein fraction represents a portion of the mixture obtained by purifying the biological preparation, in which portion the determined total concentration of proteins is greater than zero.

The hydrolytic activity of the lovastatin esterase enzyme towards lovastatin and salts thereof represents a capability of hydrolysing lovastatin and salts thereof to form the above-defined triol.

The hydrolytic activity of the lovastatin esterase enzyme, towards of simvastatin and salts thereof represents a capability of hydrolysing simvastatin and salts thereof to form the above-defined triol.

The hydrolytic activity of the lovastatin esterase enzyme towards lovastatin and salts thereof is at least by one order of magnitude higher than the hydrolytic activity of the lovastatin esterase enzyme towards simvastatin and salts thereof. This activity may be altered following immobilization of the lovastatin esterase enzyme on a solid support, particularly on a solid support insoluble in water. Without limiting the scope of the invention by theoretical considerations, one may suppose that because of chemical binding the above-defined enzyme at the surface of the solid phase, modifications of the spatial configuration of the enzyme may occur, thus altering the relative localisation of the enzyme active sites. Because of that, the hydrolytic activity of the lovastatin esterase enzyme, as well as the enzyme activity following immobilization on a solid support may differ from the activity of the enzyme in an aqueous mixture. Such a difference manifests itself in a loss of selectivity of the hydrolytic activity following immobilization on a solid support and/or in decreasing the activity.

It was unexpectedly found that the particular combination of the solid support insoluble in water and the at least difunctional coupling reagent allows obtaining the enzyme immobilized on a solid support insoluble in water, that exhibits the hydrolytic activity towards lovastatin and salts thereof at least 5 times higher, in the presence of simvastatin and/or salts thereof, than towards simvastatin and salts thereof, where the enzyme is sufficiently active to be applied for purifying and/or isolating simvastatin from the mixture with lovastatin.

By the process according to the invention, the lovastatin esterase enzyme is immobilized on the polygalactose bed, such as agarose (preferably Sepharose B4), using the at least difunctional reagent of the formula

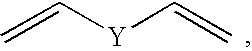

wherein Y represents —SO₂— or —SO₂—(CHR)ₙ—SO₂—, where n represents an integer of from 1 to 18, and R represents a hydrogen atom or C₁₋₆ alkyl, or Y represents —SO₂—Ar—SO₂—, where Ar represents a divalent aryl radical formed by displacing two hydrogen atoms directly bound to the aromatic ring carbon atoms, the divalent aryl radical optionally bearing C₁₋₆ alkyl substituents. Advantageously, the reagent activating the solid support is the compound of the formula

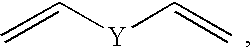

wherein Y represents —SO₂—, i.e., divinylsulphone.

The process of activation of the polygalactose solid support, according to the invention, consists in contacting the support with the compound of the formula

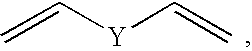

preferably divinylsulphone, what results in formation of the activated solid support. Then the mixture containing the lovastatin esterase enzyme, prepared according to the known procedure, using the buffer, is contacted with the activated solid support with mechanical agitation, over a period sufficient to immobilize the enzyme on a solid support. Following the immobilization, the obtained biocatalyst is separated by filtration, preferably washed with water and/or a buffer. The immobilized enzyme according to the invention is then used in the processes of purification and/or isolation of simvastatin.

The enzyme immobilized on a solid support using divinylsulphone as a coupling reagent was used in the hydrolysis reaction of a mixture of statins containing 15% lovastatin and 85% simvastatin. This is a typical mixture obtained in the process for preparing simvastatin from lovastatin via the chemical synthesis. The flow reactor presented in the cross-sectional view in FIG. 1 has been designed and manufactured for carrying out the reaction. The flow reactor according to the invention comprises a body 1 of the reactor with an inner space 2 connected to a fluid inlet 3 and connected to a fluid outlet 4. The inner space 2 is filled with the bed 5 containing the immobilized enzyme. The hydrolysis process was carried out continuously by feeding the solution of a mixture of the ammonium salts of lovastatin and simvastatin via the fluid inlet 3 and receiving the efflux from the fluid outlet 4. The consecutive portions of the efflux were analysed by HPLC, proving that the concentration of the simvastatin salt was not altered during the process more than by 1%, with relation to the initial concentration. The degree of hydrolysis of the lovastatin ammonium salt was at least 87%. The conversion level was not altered during the process conducted continuously for 120 hours, what proved that the flow reactor according to the invention, with the bed 5 containing the immobilized lovastatin esterase enzyme, could be effectively used in the process of manufacturing, isolating and/or purifying simvastatin from the mixture of statins containing simvastatin and lovastatin.

The technological stability of the reactor according to the invention was examined by repeating the experiment concerning the hydrolysis of the mixture of statins containing 15% lovastatin and 85% simvastatin, after a 6-month storage of the reactor at +4° C. The hydrolysis process was conducted continuously by feeding the solution of a mixture of the ammonium salts of lovastatin and simvastatin via the fluid inlet 3, and receiving the efflux from the fluid outlet 4. The consecutive portions of the efflux were analysed by HPLC, proving that the concentration of the simvastatin salt was not altered during the process more than by 1%, with relation to the initial concentration. The degree of hydrolysis of the lovastatin ammonium salt was at least 96%. The data presented in the table 3 show that the storage time of the flow reactor does not influence significantly on its performance. Within 25 hours, the conversion of lovastatin was practically unchanged, what proved that the immobilized enzyme according to the invention retained its activity as well as the selectivity of hydrolysis of the lovastatin salts in the presence of the simvastatin salts. Therefore, immobilization of the enzyme on a solid support provides a stable biocatalyst having the stability sufficient for using in the process of manufacturing, isolating and/or purifying simvastatin.

In another embodiment of the process for immobilization of the enzyme on a solid support, according to the invention, the solid support insoluble in water, such as a silica gel modified with an amino-C₁₋₆alkyl-tri(C₁₋₆alkoxy)silane, preferably a silica gel modified with aminopropylsilyl groups, was activated. The at least difunctional activating reagent, being preferably a derivative of cyanuric acid (1,3,5-triazine-2,4,6-triol derivative), such as a cyanuric halide, or wherein at least two hydroxy groups were replaced with a halogen substituent or an O-sulphonate group, such as a cyanuric halide and/or cyanuric acid O-sulphonate, was used for the activation.

The silica gel modified with an amino-C₁₋₆alkyl-tri(C₁₋₆alkoxy)silane, was activated by the treatment with a solution of the derivative of cyanuric acid, followed by suspending in a buffer and adding the mixture containing the lovastatin esterase enzyme, with mechanical agitation, over a period sufficient to immobilize the enzyme on a solid support. The immobilization was conducted using the non-purified enzyme as well as the enzyme purified by chromatography. By using aminopropylsilanized silica gel as the preferred solid support and cyanuric chloride as the preferred coupling reagent, it was found that the use of the purified enzyme affords a higher efficiency of an enzyme immobilization. Moreover, the immobilized enzyme according to the invention, that is obtained using the non-purified enzyme, exhibits an activity lower than the immobilized enzyme according to the invention, that is obtained using the purified enzyme.

In another embodiment of the process for immobilization of the enzyme according to the invention, the solid support insoluble in water, such as the cellulose modified with di-(C₁₋₆alkyl)amino-C₁₋₆alkyl groups, is activated. An example of such a solid support is diethylaminoethylcellulose. The at least difunctional activating reagent, that is preferably a derivative of cyanuric acid (1,3,5-triazine-2,4,6-triol derivative), wherein at least two hydroxy groups are replaced with a halogen substituent or an O-sulphonate group, such as a cyanuric halide and/or cyanuric acid O-sulphonate, is used for activation.

The cellulose modified with di-(C₁₋₆alkyl)amino-C₁₋₆alkyl groups was activated using a solution of the derivative of cyanuric acid, optionally in the presence of a base, to yield the activated solid support. The solid support was suspended in a buffer and the mixture containing the lovastatin esterase enzyme was added, with mechanical agitation, over a period sufficient to immobilize the enzyme on a solid support. The immobilized enzyme according to the invention is then used in the processes of preparation, isolation and/or purification of simvastatin.

The enzyme immobilized on cellulose modified with diethylaminoethyl groups, using cyanuric chloride as a coupling reagent, was used in the hydrolysis of a mixture of statins, a mixture of ammonium salts of lovastatin and simvastatin, to give a very high conversion (>99%) of the lovastatin salt into the triol within several hours. The enzyme immobilized on a solid support diethylaminoethylcellulose, having even higher activity, was obtained by using the activated solid support diethylaminoethylcellulose, that was previously annealed in an autoclave.

The obtained immobilized enzyme according to the invention was used as a bed for the reactor according to the invention, as presented in FIG. 1. The reactor was placed in a thermostatted jacket having the temperature stabilised at 28° C. The process of hydrolysis was carried out continuously supplying the solution of a mixture of lovastatin and simvastatin ammonium salts via the fluid inlet 3 and collecting the efflux from the fluid outlet 4. The consecutive portions of the efflux were analysed by HPLC every 4 hours. During the operation of the reactor, the flow rate was modified and the lovastatin conversion changes were recorded. It was found that a reduction of the conversion of lovastatin occurs for the flow rates exceeding the limiting flow rate (i.e., the maximum flow rate at which the conversion of lovastatin is 100%). For the flow rates higher than the limiting flow rate (when the conversions are significantly lower than at the limiting flow rate), a change of the conversion of lovastatin with respect to temperature of the reactor was determined. The measurements were taken for a series of temperatures. The results are presented in the diagram (FIG. 2).

At 37° C., the lovastatin esterase enzyme immobilized on a solid support insoluble in water according to the invention was found to exhibit the highest activity (FIG. 2).

The technical solution according to the invention is presented in more detail by the following examples. Throughout the following examples, the term the “LE enzyme” represents the lovastatin esterase enzyme.

### EXAMPLES

### Example 1

### Isolation and Purification of the Enzyme

The LE enzyme is produced by the fungus Clonostachys compactiuscula deposited under the number ATCC 38009. The fungus was harvested according to the procedure described in the literature (U.S. Pat. No. 5,223,415).

1a. Extraction of the LE Enzyme from the Mycelium

The mycelium of Clonostachys compactiuscula (59 g) was triturated with glass beads (0.4 g) in liquid nitrogen for 8 hours, then it was extracted with the phosphate buffer (60 mL, pH=6.5), and the solid was centrifuged off (12000 rpm; 10 min; 4° C.). The supernatant was separated and the extraction procedure with buffer (40 mL) was repeated. The supernatants were combined and filtered via a qualitative filter to yield 110 mL of the filtrate, denoted as the supernatant X1 (Cₚᵣₒₜₑᵢₙ=4180 μg/mL) in the following description.

1b. Purification of the Lovastatin Esterase by Salting Out the Active Protein Fraction

To the supernatant X1 (20 mL), ammonium sulphate was added to achieve the concentration of 40% (4.62 g, 1 hour, 0° C.). The solution was then left for 1 hour (0° C.), and centrifuged (12000 rpm; 20 min; 4° C.). The supernatant was separated and ammonium sulphate was added to achieve the concentration of 85% (6 g; 1 hour; 0° C.), and centrifuged (12000 rpm; 20 min; 4° C.). The resulting precipitate was collected and dissolved in a phosphate buffer (pH 7.8; 20 mM; 0.5 mol NaCl) to give 0.72 mL of the LE solution, named in the following as the solution X2 (Cₚᵣₒₜₑᵢₙ=980 μg/mL) having the specific activity of 6.62 μmol/min·mg.

1c. Purifying the Lovastatin Esterase by Chromatography

A hydrophobic interaction column packed with Phenyl Sepharose 6-fast flow (26 mL) was subjected to equilibration with a phosphate buffer (pH=6.5; 1 mL/min; 5 hours). Then the solution X2 (15 mL) was fed to the column, that was eluted with a phosphate buffer (pH=6.5; 1.4 mL/min), redistilled water (1.4 mL/min), collecting the fractions containing the LE enzyme. Following the separation, the column was rinsed with a phosphate buffer (pH=6.5; 1.4 mL/min; 30 min) again. The active fractions containing the LE enzyme were pooled and the carbonate buffer (1 mL, pH=9.4; 50 mmol) was added to yield 12.5 mL of the solution (Cₚᵣₒₜₑᵢₙ=21 μg/mL) named further the solution X3, having a specific activity of 290 μmol/mg·min (assay by HPLC).

### Example 2

### Immobilization of the LE Enzyme on Sepharose B4 (Agarose Gel)

2a. Activation of the Solid Support

Sepharose B4 (5 mL) was washed with water (20×5 mL), a phosphate buffer ( 1/15 M, pH=5.6, 20×5 mL), water (20×5 mL), a carbonate buffer (50 mmol, pH=9.6, 20×5 mL). A 100 mg sample of the obtained bed was dried and analysed.

IR (KBr): 3436 (35%), 2901 (60%), 1650 (55%), 1474 (55%), 1415 (55%), 1376 (50%), 1307 (55%), 1250 (55%), 1190 (35%), 1157 (35%), 1071 (30%), 1042(%), 988 (55%), 966 (50%), 931 (40%), 891 (45%), 870 (60%), 788 (60%), 772 (55%), 741 (50%), 716 (50%), 693 (50%), 538 (50%), 483 (50%) cm⁻¹.

Elementary analysis: found: C, 41.61%; H, 6.47%; N, 0.0%.

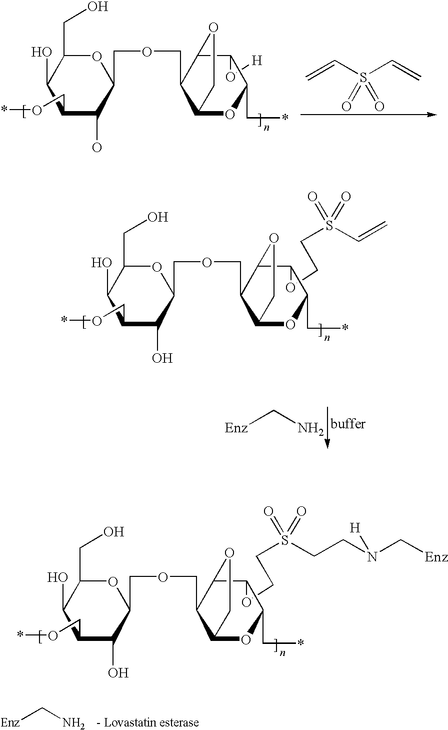

The obtained solid support was suspended in a carbonate buffer (10 mL, 50 mmol pH=9.6), divinylsulphone (1 mL, 10 mmol) was added and the mixture was shaken for 70 min. The solid support was washed with water (20×5 mL), a phosphate buffer (20×5 mL, 1/15 M, pH=5.6), water (20×5 mL), and a carbonate buffer (20×5 mL, 50 mmol, pH=9.6). A 100 mg sample of the obtained solid support was dried and analysed.

IR (KBr): 3436 (40%), 2902 (65%), 1651 (55%), 1475 (60%), 1413 (55%), 1377 (50%), 1302 (60%), 1251 (60%), 1183 (40%), 1157 (40%), 1076 (30%), 1044 (30%), 988 (60%), 966 (60%), 931 (50%), 891 (60%), 772 (70%), 741 (70%), 715 (70%) cm⁻¹.

Elementary analysis: found: C, 43.50%; H, 6.76%; N, 0%; S, 0.88%.

2b. Immobilization of the LE Enzyme on a Solid Support

To the activated solid support, washed with water (20×5 mL), a carbonate buffer (5 mL, 50 mM pH=9.6), and the supernatant X1 extracted from the microorganism Clonostachys compactiuscula, (Cₚᵣₒₜₑᵢₙ=21178 μg/mL, activity=13.2 μmol/mg·mL) in a carbonate buffer (0.5 mL, 50 mmol, pH=9.6) were added, and the mixture was shaken for 18 hours. The solid support was separated by filtration, suspended in a glycine buffer (10 mL, 50 mmol, pH=9.6), shaken for 2 hours, washed with water (20×5 mL), a phosphate buffer (20×5 mL, 1/15 mol, pH=5.6), water again (20×5 mL), and a glycine buffer (20×5 mL, 50 mmol, pH=9.6). 910 μg of the protein was immobilized.

### Example 3

### Hydrolysis of a Mixture of Ammonium Salts of the Statins with the LE Enzyme Immobilized on a Solid Support, in a Batch Process

The LE enzyme immobilised on a solid support Sepharose B4 (obtained in Example 2b; dry weight of 9.2 mg) was suspended in a mixture of ammonium salts of the statins (1 mL, Cₛₜₐₜᵢₙₛ=0.8 mg/mL) dissolved in a glycine buffer (10 mL, 50 mmol, pH=9.6). The suspension was shaken for 90 minutes at 30° C. The immobilized LE enzyme was separated by filtration, and the hydrolytic activity towards the lovastatin ammonium salt (the hydrolytic activity L) and towards the simvastatin ammonium salt (the hydrolytic activity S), and also the corresponding conversions, were determined by HPLC analysis. The immobilized LE enzyme was washed with water (5×5 mL) and the hydrolysis batch process was repeated. After 4 repetitions, 5.7 mg of the immobilised LE enzyme (after drying to a constant weight) were recovered. The results of the analysis for this biocatalyst are presented in Table 1.

TABLE 1

| | | The hydrolytic | | The hydrolytic | |
|---|---|---|---|---|---|
| | Conversion L | activity L | Conversion S | activity S | Selectivity |
| Repetition | (%) | (μM/mg · mL) | (%) | (μM/g · mL) | L/S |
| The native | | 10.71 | | 0.06 | >100 |
| LE enzyme | | | | | |
| 1 | 33.4 | 4.66 | 8.3 | 0.81 | 5.75 |
| 2 | 31.1 | 4.36 | 9.9 | 0.97 | 4.49 |
| 3 | 28.5 | 3.97 | 9.3 | 0.91 | 4.36 |
| 4 | 19.4 | 2.72 | 9.0 | 0.88 | 3.09 |

### Example 4

### Hydrolysis of a Mixture of Ammonium Salts of the Statins Using the Flow Reactor

Using 10 mL of Sepharose B4 and the solution X3 of the purified LE enzyme from Example 1c (Cₚᵣₒₜₑᵢₙ=46.1 μg/mL, activity=56.1 nmol/mg·min), immobilization was carried out according to the procedure described in Example 3. 123.1 μg of total protein were immobilized.

The resulting biocatalyst was employed for preparing the flow reactor.

The body of the reactor was a tube of acid-resistant steel (such as used for making high pressure chromatographic columns), containing a perforated plate transverse to the column axis upstream the fluid outlet, that supported the bed. In the Example, a chromatographic column having a diameter of 3.7 mm and a length of 149 mm (inner space volume 1.6 mL) was used, which was protected with a nut and a frit at the bottom, and extended by a ferrule at the top. The above-prepared immobilized enzyme as a suspension in a glycine buffer (pH=9.4, 20 mM, 5 mL) was introduced into this set. The bed was formed by passing the eluent (flow rate: 0.4 mL/min, glycine buffer pH=9.4, 20 mM, 5 mL). Formation of the bed was deemed complete after achieving stabilisation of the pressure and stabilisation of the absorbance (as determined by an UV detector at λ=254 nm). Then the extending ferrule was removed and the exposed bed was secured with a nut and a frit. The bed in the reactor was subjected to conditioning (flow rate: 0.4 mL/min) by passing a glycine buffer (pH=9.4, 20 mM, 5 mL).

A mixture of the ammonium salts of statins, containing 15% lovastatin and 85% simvastatin, was then introduced into the reactor at a flow rate of 0.4 mL/min. The efflux was sampled every 6 hours and analysed by HPLC. The analysis results, recalculated to the degree of hydrolysis, are summarised in Table 2. During the whole process, the concentration of the simvastatin salt varied not more than by 1%. The lovastatin ammonium salt was hydrolysed in about 87%. This conversion was unchanged over 120 hours.

TABLE 2
Hydrolysis of the mixture of statins in a flow reactor with the
bed containing the LE enzyme immobilized on a solid support

| Time of | | | |
|---|---|---|---|
| run elapsed | | | |
| before sampling | | | Selectivity |
| the efflux | Conversion | Conversion | (conversion L/ |
| (hours) | L (%) | S (%) | conversion S) |
| 0 | 87.9 | <0.1 | >100 |
| 6 | 86.6 | <0.1 | >100 |
| 12 | 86.1 | <0.1 | >100 |
| 18 | 87.4 | <0.1 | >100 |
| 24 | 87.0 | <0.1 | >100 |
| 30 | 86.8 | <0.1 | >100 |
| 36 | 86.6 | <0.1 | >100 |
| 42 | 86.9 | <0.1 | >100 |
| 48 | 86.9 | <0.1 | >100 |
| 54 | 87.7 | <0.1 | >100 |
| 60 | 87.1 | <0.1 | >100 |
| 66 | 86.6 | <0.1 | >100 |
| 72 | 88.8 | <0.1 | >100 |
| 78 | 88.4 | <0.1 | >100 |
| 84 | 87.9 | <0.1 | >100 |
| 90 | 87.0 | <0.1 | >100 |
| 96 | 90.6 | <0.1 | >100 |
| 102 | 87.2 | <0.1 | >100 |
| 108 | 87.6 | <0.1 | >100 |
| 114 | 86.6 | <0.1 | >100 |
| 120 | 87.6 | <0.1 | >100 |
| 126 | 87.9 | <0.1 | >100 |

### Example 5

### Estimation of Technological Stability of the Reactor with the Bed Containing the LE Enzyme Immobilized on a Solid Support

The inlet and the outlet of the flow reactor used in Example 4 were secured with tightly fixed caps, a then the reactor was stored for 6 months at 4° C. After stabilising, the reactor was used again to hydrolyse the mixture of ammonium salts of statins, following the procedure of Example 4. The results of conversion of the lovastatin salt and the simvastatin salt are summarised in Table 3.

TABLE 3
Hydrolysis of the mixture of statins in a flow reactor with the
bed containing the LE enzyme immobilized on a solid support
(the reactor reused after a 6-month storage at 4° C.)

| Time of | | | |
|---|---|---|---|
| run elapsed | | | |
| before sampling | | | Selectivity |
| the efflux | Conversion | Conversion | (conversion L/ |
| (hours) | L (%) | S (%) | conversion S) |
| 0 | 97.1 | <0.1 | >100 |
| 2 | 97.7 | <0.1 | >100 |
| 3 | 98.0 | <0.1 | >100 |
| 5 | 97.6 | <0.1 | >100 |
| 7 | 97.3 | <0.1 | >100 |
| 9 | 97.5 | <0.1 | >100 |
| 11 | 96.6 | <0.1 | >100 |
| 13 | 96.8 | <0.1 | >100 |
| 15 | 96.4 | <0.1 | >100 |
| 17 | 96.2 | <0.1 | >100 |
| 19 | 96.1 | <0.1 | >100 |
| 21 | 96.0 | <0.1 | >100 |
| 23 | 96.7 | <0.1 | >100 |
| 25 | 96.6 | <0.1 | >100 |

### Example 6

### Immobilization of the LE Enzyme on a Solid Support (Silica Gel) Using Cyanuric Chloride

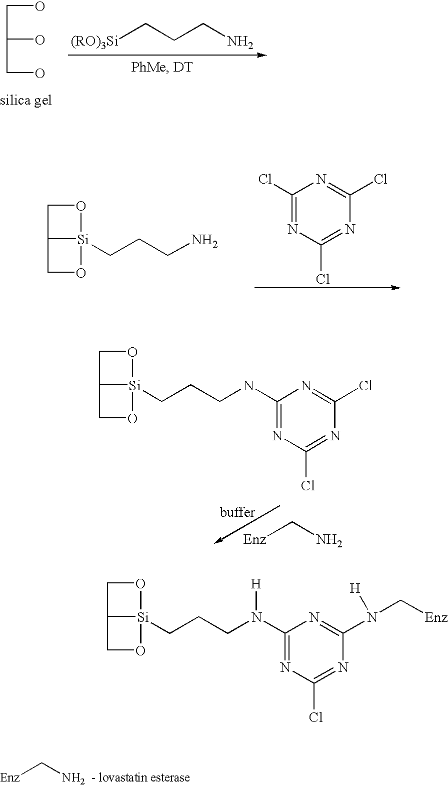

6a. Modifying the Silica Gel

Silica gel (50 g; 200-300 mesh) was placed on a sintered-glass funnel and rinsed with nitric acid (5%, 15 mL) and water (15 mL). A portion of the gel (30 g) was then flooded with toluene (210 mL) and dried by distilling off the azeotropic mixture (water-toluene, 110° C.; 4 hours). After cooling to 80° C., (3-aminopropyl)trimethoxysilane (6 mL, 34.3 mmol) was added, and the mixture was heated at 80° C. for 2 hours. The obtained solid support was filtered off and rinsed with toluene (20 mL), hexane (20 mL), tetrahydrofuran (20 mL) and toluene again (20 mL). The material was dried at 80° C. until the constant weight to, yield 24 g of the solid support. Elementary analysis: found: C, 4.99%; H, 1.12%; N, 1.69%.

6b. Activation of the Solid Support and Immobilization of the LE Enzyme

The aminopropylsilylated silica gel obtained in Example 6a (250 mg, 200-300 mesh, containing 1 mmol of amino groups per 1 g of the solid support) was added to the solution of cyanuric chloride (62.8 mg, 0.34 mmol) in a mixture of dioxane (5 mL) and toluene (1 mL), at 12° C., and shaken on a shaker for 3 hours. The solid support was filtered off, rinsed with toluene (15 mL) and acetone (15 mL), and dried under reduced pressure. The supernatant X1 obtained in Example 1a (11.5 mL, Cₚᵣₒₜₑᵢₙ=4180 μg/mL) was added to the thus-prepared solid support and shaken on a shaker for 2.5 hours. The obtained biocatalyst (the LE enzyme on a solid support) was filtered off and washed with a carbonate buffer (50 mmol, pH=9.4; 5×5 mL) to yield the immobilized enzyme and the filtrate. The immobilisation yield was 16%.

The obtained biocatalyst was employed for the hydrolysis of a mixture of ammonium salts of the statins giving a conversion of lovastatin of 7.1% over 90 minutes. The selectivity of the obtained biocatalyst was >100. The hydrolysis reaction carried out using the non-immobilized enzyme (remaining in the filtrate) had a yield of 11.5%.

### Example 7

### Immobilization of the LE Enzyme on a Solid Support (Modified Silica Gel) Using Cyanuric Chloride

The aminopropylsilylated silica gel obtained in Example 6a (500 mg, 200-300 mesh, containing 1 mmol of amino groups per 1 g of the bed) was added to the solution of cyanuric chloride (125.9 mg, 0.68 mmol) in a mixture of dioxane (5 mL) and toluene (1 mL), at 9° C., and shaken on a shaker for 3 hours. Then the activated solid support was filtered off, rinsed with toluene (25 mL) and acetone (25 mL), and dried under reduced pressure. A portion of the activated solid support (250 mg) was suspended in the solution X2 obtained in Example 1c (11.5 mL, Cₚᵣₒₜₑᵢₙ=21 μg/mL) and shaken on a shaker for 2.5 hours. The obtained biocatalyst (the LE enzyme on a solid support) was filtered off and washed with a carbonate buffer (50 mmol, pH=9.4, 5×5 mL) to yield the immobilized enzyme and the filtrate. The immobilisation yield was 52%.

The obtained biocatalyst was employed for the hydrolysis of a mixture of ammonium salts of the statins giving the conversion of lovastatin of 34.1% over 90 minutes. The selectivity of the biocatalyst was >100. The same reaction carried out using the filtrate does not proceed at all, what testifies to complete immobilisation of the LE on a solid support.

### Example 8

### Immobilization of the LE Enzyme on a Solid Support (Modified Silica Gel) Using Cyanuric Chloride

The aminopropylsilylated silica gel obtained in Example 6a (500 mg, 200-300 mesh, containing 1 mmol of amino groups per 1 g of the bed) was added to the solution of cyanuric chloride (9.3 mg, 0.05 mmol) in a mixture of dioxane (5 mL) and toluene (1 mL), at 9° C., and shaken on a shaker for 3 hours. The activated solid support was filtered off, rinsed with toluene (25 mL) and acetone (25 mL), and dried under reduced pressure. Then the solid support (250 mg) was suspended in the solution X3 of the LE enzyme obtained in Example 1c (11.5 mL, Cₚᵣₒₜₑᵢₙ=21 μg/mL) and shaken on a shaker for 2.5 hours. The obtained biocatalyst was filtered off and washed with a carbonate buffer (50 mmol, pH=9.4, 5×5 mL) to yield the immobilized enzyme and a filtrate. The immobilisation yield was 52%.

The obtained biocatalyst was employed for the hydrolysis of a mixture of ammonium salts of the statins, giving the conversion of lovastatin of 16.3% over 90 minutes. The selectivity of the biocatalyst was >100. The same reaction carried out using the filtrate does not proceed at all, what testifies to complete immobilisation of the LE on a solid support. Following the above hydrolysis of a mixture of ammonium salts of the statins, the biocatalyst was rinsed with a carbonate buffer (50 mmol, pH=9.4, 5×5 mL) and stored for 7 days at 4° C. Then the biocatalyst was used again for the hydrolysis of a mixture of ammonium salts of the statins giving the conversion of lovastatin of 26.1% over 90 minutes. The selectivity of the biocatalyst was >100.

### Example 9

### Immobilization of the LE Enzyme on a Solid Support (diethylaminoethylcellulose) Using Cyanuric Chloride

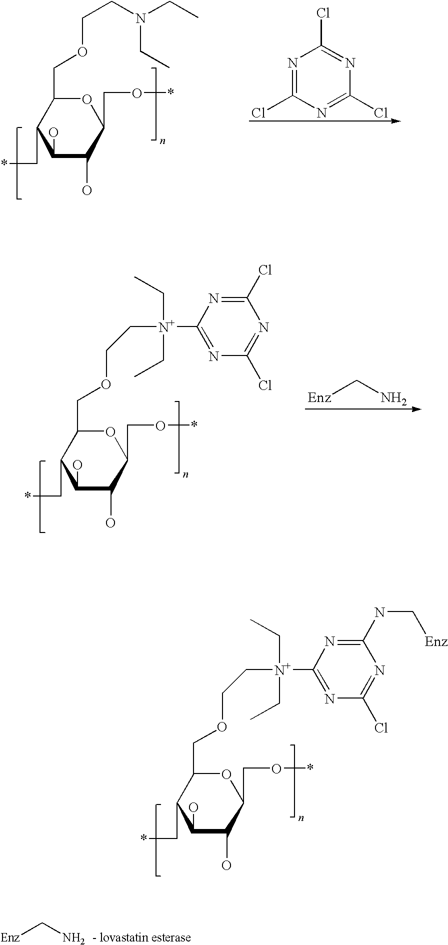

Diethylaminoethylcellulose (0.5 g) was washed with water (10 mL), suspended in a sodium hydroxide solution (1 mol, 10 mL), shaken (15 min, 250 rpm), filtered, washed with water (10 mL) and suspended in dioxane (10 mL). A solution of cyanuric chloride (1 g, 5.4 mmol) in toluene (10 mL) was added to the obtained suspension. The suspension was shaken (30 min, 250 rpm), filtered, the precipitate was rinsed with dioxane (2×10 mL), a mixture of acetic acid/water/dioxane (2/2/4 mL), water (10 mL), acetone (2×10 mL), and dried under reduced pressure (0.2 tor). The solution X3 obtained in Example 1c (13 mL, specific activity 313 μmol·min⁻¹·mg⁻¹) was added to the activated solid support. The suspension was shaken on a shaker for 18 hours (250 rpm), a then the biocatalyst was filtered off, washed with water (2×10 mL), and carbonate buffer (2×10 mL, 50 mmol, pH=9.4). The immobilisation yield was 37.2%. The activity of the obtained immobilized enzyme was checked by hydrolysing 8 mL of a mixture of ammonium salts of the statins, to obtain the specific activity value of 311 μmol·min⁻¹·mg⁻¹. The selectivity of the biocatalyst was >100.

The above-obtained immobilized LE enzyme was added to the solution of ammonium salts of simvastatin (294 mg, 0.67 mmol) and lovastatin (21.7 mg, 0.05 mmol) in a carbonate buffer (150 mL, 50 mmol, pH=9.4) at 30° C., with magnetic stirring (250 rpm). The reaction progress and selectivity of the hydrolysis are analysed by HPLC.

TABLE 4
Conversion of the salt of lovastatin in the reaction catalysed with
the LE enzyme immobilized on diethylaminoethylcellulose
activated with cyanuric chloride

| Time of | Conversion | Selectivity |
|---|---|---|
| hydrolysis (min) | (%) | L/S |
| 0 | 0 | |
| 10 | 11.0 | >100 |
| 15 | 14.9 | >100 |
| 22 | 18.2 | >100 |
| 37 | 30.5 | >100 |
| 57 | 39.6 | >100 |
| 69 | 44.5 | >100 |
| 360 | 100 | >100 |

### Example 10

### Immobilization of the LE Enzyme on a Solid Support (diethylaminoethylcellulose) Using Cyanuric Chloride

The procedure described in Example 9 was followed using the previously autoclaved diethylaminoethylcellulose without initial purification and the LE enzyme having the specific activity (L) of 61.2 μmol·min⁻¹·mg⁻¹.

The obtained immobilized LE enzyme was used for the preparative hydrolysis of the mixture of ammonium salts of the statins, according to the procedure described in Example 9.

TABLE 5
Conversion of the salt of lovastatin in the reaction catalysed with
the LE enzyme immobilized on diethylaminoethylcellulose
activated with cyanuric chloride

| | |
|---|---|
| Time of | Conversion of lovastatin (%) |

| hydrolysis | Autoclaved | Non-autoclaved | Selectivity |
|---|---|---|---|
| (hours) | cellulose | cellulose | L/S |
| 0 | 0 | 0 | |
| 1.3 | 15.5 | 11.6 | >100 |
| 19.3 | 85.7 | 62.8 | >100 |

It was found that autoclaving of the solid support prior to immobilization of the LE enzyme increased the activity of the biocatalyst by 35%, compared to the enzyme LE immobilized on a non-autoclaved cellulose. The selectivity of the biocatalyst was >100.

### Example 11

### Hydrolysis of a Mixture of Ammonium Salts of the Statins Using the Flow Reactor

The body of a chromatographic column (0.58 cm in diameter, 9.6 cm length, 2.5 mL inner space volume) was secured with a frit at one end. An extending ferrule was put on the other end. The immobilized LE enzyme obtained in Example 9, as a suspension in a carbonate buffer (10 mL, 50 mmol, pH=9.4, 0.2 mg/mL NaN₃, 0.3 mg/mL EDTA) was introduced into the obtained set. After dribbling the buffer from the frit, the extending ferrule was removed and the exposed bed was secured with the second frit (the amount of the bed corresponded to the weight of product obtained from 0.8 g of diethylaminoethylcellulose and contains 0.62 mg of the immobilised protein). The bed in the reactor was formed with a flow rate of a carbonate buffer (0.4 mL/min, 50 mmol, pH=9.4, 0.2 mg/mL NaN₃, 0.3 mg/mL EDTA) for one hour. Then the solution of the ammonium salts of simvastatin (0.74 mg/mL) and lovastatin (0.06 mg/mL) was pumped through the reactor thermostatted at 28° C. Every 4 hours, the contents of the ammonium salts of the statins in the efflux was determined by HPLC.

The experiments were carried out for the flow rates varying from 0.175 mL/min to 0.2 mL/min.

TABLE 6
Effect of the flow rate on the conversion of the lovastatin salt in
the reaction catalysed with the LE enzyme immobilized on
diethylaminoethylcellulose activated with cyanuric chloride

| Flow rate | Conversion of | Selectivity |
|---|---|---|
| (mL/min) | lovastatin (%) | L/S |
| 0.175 | 100 | >100 |
| 0.185 | 92 | >100 |
| 0.2 | 86 | >100 |

By controlling the temperature of the thermostatted column, a thermal dependence of the conversion of lovastatin was determined.

TABLE 7
Effect of temperature on the conversion of lovastatin salt in the
reaction catalysed with LE enzyme immobilised on
diethylaminoethylcellulose activated with cyanuric chloride

| Temperature | Conversion of | Selectivity |
|---|---|---|
| (° C.) | lovastatin (%) | L/S |
| 28 | 52.1 | >100 |
| 30 | 50.6 | >100 |
| 32 | 50.8 | >100 |
| 34 | 58.0 | >100 |
| 36 | 61.1 | >100 |
| 38 | 59.9 | >100 |
| 40 | 41.3 | >100 |
| 42 | 31.2 | >100 |
| 44 | 20.6 | >100 |

The results presented in Table 7 indicate that the immobilised LE enzyme has the highest activity at 37° C.

### REFERENCE EXAMPLES

### Reference Example 1

### Immobilization of the LE Enzyme on a Modified Silica Gel

10 g of silica gel were added to the mixture of γ-aminopropyltriethoxysilane and toluene (2% by volume, 100 mL), and the mixture was refluxed for 10 hours. The gel was then filtered off, rinsed with acetone (2×5 mL), water (5 mL), acetone again (2×5 mL) and dried with dry air for 10 hours. The thus-prepared gel (100 mg) was suspended in a carbonate buffer (2 mL, 50 mM, pH 9.4) and divinylsulphone (0.25 mL, 2.50 mmol) was added at 20° C. After 30 minutes, the solid support was separated off and dried in vacuo for 2 hours. This was then rinsed with distilled water (4×20 mL), suspended in a carbonate buffer (1 mL, 50 mM, pH 9.4) and soaked with a solution of the LE enzyme (1 mL, 0.225 mg/mL). The mixture was left for 20 hours at 20° C. The solid support with the LE enzyme deposited thereon was filtered off and rinsed with distilled water (2×10 mL).

### Reference Example 2

### Immobilization of the LE Enzyme on Wool

Small pieces of wool (50 mg) were suspended in a carbonate buffer (2 mL, 50 mM, pH 9.4) and then divinylsulphone (0.25 mL, 2.5 mmol) was added at 20° C. After 30 minutes, the wool was separated, dried in vacuo (2 hours) and rinsed with distilled water (4×20 mL). The solid support was suspended in a carbonate buffer (1 mL, 50 mM, pH 9.4) again, soaked with a solution of the LE enzyme (1 mL, 0.225 mg/mL), and left for 10 hours at the room temperature. The solid support with the LE enzyme deposited thereon was filtered off and rinsed with distilled water (2×10 mL).

### Reference Example 3

### Immobilization of the LE Enzyme by a Sol-Gel Method

A solution of the LE enzyme (0.5 mL, 0.225 mg/mL) was mixed with a Tris buffer (0.5 mL, 0.1 M, pH=7.5) and left on a shaker (10 minutes). Then the aqueous solution of polyvinyl alcohol (100 μL, 4% by volume), sodium fluoride (50 μL, 1 M) and isopropyl alcohol (100 μL) were added. After 5 minutes, isobutyltrimethoxysilane (2.5 mmol) and TMOS (0.5 mmol, 74 μL; 76 mg) were added and the mixture was left on a shaker. The mixture was allowed to dry in an open vessel overnight at the room temperature, and then isopropyl alcohol (10 mL) was added. The gel was separated and washed with water (10 mL), isopropyl alcohol, (10 mL) and n-pentane (10 mL). The obtained gel was crushed and left to dry overnight at the room temperature.

### Reference Example 4

### Immobilization of the LE Enzyme by a Sol-Gel Method

The method according to the Reference Example 3 was used, with a modification consisting in adding Tween® 80 (0.1 mL) to the solution of the enzyme.

### Reference Example 5

### Immobilization of the LE Enzyme by a Sol-Gel Method

The method according to the Reference Example 3 was used, with a modification consisting in adding the lovastatin ammonium salt (50 mg) to the solution of the enzyme.

### Reference Example 6

### Immobilization of the LE Enzyme on Eupergit® C

The freeze-dried LE enzyme (17 mg, protein content of 2.6%) was dissolved in a carbonate buffer (1 mL, 50 mM, pH 9.4) and Eupergit® C (50 mg, a copolymer of methacrylamide and glycidyl methacrylate crosslinked with N,N′-methylenebisacrylamide) was added to the solution and left for 1 day at the room temperature. Then immobilized enzyme was filtered off and rinsed with distilled water (2×10 mL).

### Reference Example 7

### Immobilization of the LE Enzyme by Encapsulation in Calcium Alginate

A solution of the purified LE enzyme (0.8 mL, 0.225 mg/mL) was mixed thoroughly with an aqueous solution of calcium alginate (8 mL, 2% wt./v.). Then, the thus-obtained solution was added dropwise to the aqueous solution of calcium chloride (10 mL, 280 mM), mixed for 20 minutes, the precipitate was filtered off, and washed with a distilled water (2×10 mL). The obtained immobilizate was stored under distilled water at 4° C.

### Reference Example 8

### Immobilization of the LE Enzyme by Encapsulation in Calcium Alginate

A solution of the purified enzyme (0.8 mL, 0.225 mg/mL) was mixed thoroughly with an aqueous solution of calcium alginate (8 mL, 2% wt./v.). Then, the thus-obtained solution was added dropwise to the aqueous solution of calcium chloride (10 mL, 280 mM), mixed for 20 minutes, the precipitate was filtered off, washed with a distilled water (2×10 mL). The obtained material was suspended in tetramethoxysilane (1.0 mL) and stirred for 15 minutes at 4° C. The whole mixture was allowed to polymerize for 12 hours. The immobilized enzyme was filtered off, washed with a distilled water (2×10 mL) and dried at the room temperature.

### Reference Example 9

The immobilized LE enzymes obtained in the Reference Examples 1-8 were added to the solution of the ammonium salts of simvastatin (294 mg, 0.67 mmol) and lovastatin (21.7 mg, 0.05 mmol) in a carbonate buffer (150 mL, 50 mmol, pH=9.4) at 30° C., with magnetic stirring (250 rpm). The reaction progress and the selectivity of the hydrolysis were analysed by HPLC. The results obtained for the materials prepared in Reference Examples 1-8 are presented in Table 8.

TABLE 8

| | Yield of | | | |
|---|---|---|---|---|
| Reference | immobilization | Specific LE activity^{b} | | |
| Example | (%)^{a} | Lovastatin | Simvastatin | Selectivity^{c} |
| The native | — | 15 | 0 | >99 |
| LE enzyme | | | | |
| 1 | 80 | <0.5 | <0.5 | N.D. |
| 2 | 54 | <0.5 | <0.5 | N.D. |
| 3 | 74 | 0.9 | 3.0 | 0.3 |
| 4 | 67 | 10 | 17.8 | 0.6 |
| 5 | 61 | 7.4 | 6.2 | 1.2 |
| 6 | 80 | 8.5 | 4.2 | 2.0 |
| 7 | N.D. | N.D. | N.D. | 3.5 |
| 8 | N.D. | <0.5 | <0.5 | N.D. |

N.D.—not determined
^{a}the yield of immobilization is determined as: an amount of the immobilized protein/total amount of the protein · 100%;
^{b}the amount of the respective substrate hydrolyzed with the enzyme within one minute per milligram of protein (μM/min · mg);
^{c}the specific LE activity for lovastatin/the specific activity LE for simvastatin.

1. The lovastatin esterase enzyme immobilized on a solid support insoluble in water, characterized in that the enzyme is covalently bound to a solid support activated with an at least difunctional coupling reagent, the combination of solid support and at least difunctional coupling agent being such, that the immobilized lovastatin esterase exhibiting at least 5 times higher the hydrolytic activity towards lovastatin and salts thereof, in the presence of simvastatin and/or salts thereof, than towards simvastatin and salts thereof.

2. (canceled)

3. (canceled)

4. The lovastatin esterase enzyme according to claim 1, characterized in that the solid support is a modified polysaccharide comprising di-(C₁₋₆alkyl)amino-C₁₋₆alkylcellulose, especially diethylaminoethylcellulose, and the at least difunctional reagent activating the solid support is cyanuric acid O-sulphonate or cyanuric halide, especially cyanuric chloride.

5. (canceled)

6. The lovastatin esterase enzyme according to claim 1, characterized in that the solid support is a modified silica gel, especially modified with amino-C₁₋₆ alkyl-tri(C₁₋₆ alkoxy)silane, especially an aminopropylsilanized silica gel, and the at least difunctional reagent activating the solid support is cyanuric acid O-sulphonate or cyanuric halide, especially cyanuric chloride.

7. The lovastatin esterase enzyme according to claim 1, characterized in that the solid support is a polygalactoside and the at least difunctional reagent activating the solid support is a compound of the formula 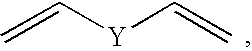
wherein Y represents —SO₂— or —SO₂—(CHR)ₙ—SO₂—, where n represents an integer of from 1 to 18, and R represents a hydrogen atom or C₁₋₆ alkyl, or Y represents —SO₂—Ar—SO₂—, where Ar represents a divalent aryl radical formed by displacing two hydrogen atoms directly bound to the aromatic ring carbon atoms, the divalent aryl radical optionally bearing C₁₋₆ alkyl substituents.

8-10. (canceled)

11. The lovastatin esterase enzyme according to claim 1, characterized in that the solid support is a polygalactoside, and the at least difunctional reagent activating the solid support is the compound of the formula 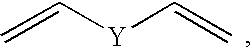
wherein Y represents —SO₂—.

12. The lovastatin esterase enzyme according to claim 1, characterized in that the solid support is diethylaminoethylcellulose, and the at least difunctional reagent activating the solid support is cyanuric chloride.

13. The lovastatin esterase enzyme according to claim 1, characterized in that the solid support is an aminopropylsilanized silica gel, and the at least difunctional reagent activating the solid support is cyanuric chloride.

14. The lovastatin esterase enzyme according to claim 1, characterized in that the enzyme is an enzyme produced by Clonostachys compactiuscula ATTC 38009, ATCC 74178.

15. A process for immobilization of the lovastatin esterase enzyme on a solid support insoluble in water, characterized in that using mechanical agitation, a cyanuric halide is contacted with a solid support comprising modified polysaccharide or modified silica gel in a solvent, the activated solid support is separated by filtration, the activated solid support is dried and suspended in an aqueous mixture containing the lovastatin esterase enzyme, until immobilization of the enzyme, the suspended material is separated by filtration, washed with a buffer and dried.

16. (canceled)

17. A process according to claim 15, characterized in that, the modified polysaccharide is a di-(C₁₋₆alkyl)amino-C₁₋₆alkylcellulose, especially diethylaminoethylcellulose.

18. (canceled)

19. A process according to claim 18, characterized in that a modified silica gel is silica gel modified with amino-C₁₋₆alkyl-tri(C₁₋₆alkoxy)silane, especially an aminopropylsilanized silica gel.

20. A process according to claim 15, characterized in that the cyanuric halide used is cyanuric chloride.

21. A process according to claim 15, characterized in that an autoclaved solid support is used.

22-24. (canceled)

25. A process according to claim 15, characterized in that the enzyme-containing aqueous solution used is a protein fraction of the material extracted from Clonostachys compactiuscula ATTC 38009, ATCC 74178.

26. A process for immobilization of the lovastatin esterase enzyme on a solid support insoluble in water, characterized in that the compound of the formula 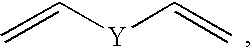
wherein Y represents —SO₂— or —SO₂—(CHR)ₙ—SO₂—, where n represents an integer of from 1 to 18, and R represents a hydrogen atom or C₁₋₆ alkyl, or Y represents —SO₂—Ar—SO₂—, where Ar represents a divalent aryl radical formed by displacing two hydrogen atoms directly bound to the aromatic ring carbon atoms, the divalent aryl radical optionally bearing C₁₋₆ alkyl substituents, is contacted with the solid polygalactose support in a solvent using mechanical agitation, the activated solid support is separated by filtration, the activated solid support is dried and suspended in an aqueous mixture containing the lovastatin esterase enzyme, the suspended material is separated by filtration, washed with a buffer and dried.

27. A process according to claim 26, characterized in that the compound of the formula 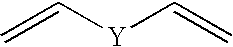
is a compound wherein Y represents —SO₂—.

28. (canceled)

29. (canceled)

30. A process according to claim 26, characterized in that the enzyme-containing aqueous solution used is a protein fraction of the material extracted from Clonostachys compactiuscula ATTC 38009, ATCC 74178.

31-36. (canceled)

37. A biocatalytic flow reactor with a bed comprising a body of the reactor with an inner space connected to the fluid inlet and connected to the fluid outlet, in which inner space there is a bed containing the lovastatin esterase enzyme immobilized on a solid support insoluble in water, characterized in that the enzyme is covalently bound to the solid support activated with an at least difunctional coupling reagent, the combination of solid support and at least difunctional coupling agent being such, that the immobilized lovastatin esterase exhibits at least 5 times higher the hydrolytic activity towards lovastatin and salts thereof in the presence of simvastatin and/or salts thereof, than towards simvastatin and salts thereof.

38-44. (canceled)

45. A biocatalytic flow reactor according to claim 37, characterized in that the solid support is a polygalactoside, and the at least difunctional reagent activating the solid support is a compound of the formula 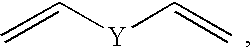
wherein Y represents —SO₂—.

46. A biocatalytic flow reactor according to claim 37, characterized in that the solid support is diethylaminoethylcellulose, and the at least difunctional reagent activating the solid support is cyanuric chloride.

47. A biocatalytic flow reactor according to claim 37, characterized in that the enzyme is an enzyme produced by Clonostachys compactiuscula ATTC 38009, ATCC 74178.

48. A process for preparation and/or purification of simvastatin comprising treating the solution of the simvastatin salt containing residual content of the lovastatin salt with the lovastatin esterase enzyme until hydrolysing lovastatin to form the triol, separating the triol, and isolating simvastatin substantially free from lovastatin, where the solution of the simvastatin salt containing residual content of the lovastatin salt is brought into a contact with the lovastatin esterase enzyme immobilized on a solid support insoluble in water, characterized in that the enzyme is covalently bound to the solid support activated with an at least difunctional coupling reagent, the combination of solid support and at least difunctional coupling agent being such, that the immobilized lovastatin esterase exhibits at least 5 times higher the hydrolytic activity towards lovastatin and salts thereof in the presence of simvastatin and/or salts thereof, than towards simvastin and salts thereof.

49-58. (canceled)

59. A process for preparation and/or purification of simvastatin according to claim 48, characterized in that the solid support is a polygalactoside, and the at least difunctional reagent activating the solid support is the compound of the formula 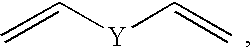
wherein Y represents —SO₂—.

60. A process for preparation and/or purification of simvastatin according to claim 48, characterized in that the solid support is diethylaminoethylcellulose, and the at least difunctional reagent activating the solid support is cyanuric chloride.

61. A process for preparation and/or purification of simvastatin according to claim 48, characterized in that the solid support is an aminopropylsilanized silica gel, and the at least difunctional reagent activating the solid support is cyanuric chloride.

62. (canceled)

63. (canceled)

64. A process for preparation and/or purification of simvastatin according to claim 48, characterized in that the enzyme is an enzyme produced by Clonostachys compactiuscula ATTC 38009, ATCC 74178.

65. The lovastatin esterase enzyme according to claim 11, characterized in that the enzyme is an enzyme produced by Clonostachys compactiuscula ATTC 38009, ATCC 74178.

66. The lovastatin esterase enzyme according to claim 12, characterized in that the enzyme is an enzyme produced by Clonostachys compactiuscula ATTC 38009, ATCC 74178.

67. The lovastatin esterase enzyme according to claim 13, characterized in that the enzyme is an enzyme produced by Clonostachys compactiuscula ATTC 38009, ATCC 74178.

68. A process according to claim 17, characterized in that an autoclaved solid support is used.

69. A process according to claim 19, characterized in that an autoclaved solid support is used.
